(19) United States
(12) Patent Application Publication
(10) Pub. No.: US 2021/0023375 A1
(43) Pub. Date: Jan. 28, 2021

(54) SYSTEMS, DEVICES AND METHODS FOR TREATMENT USING SPINAL CORD STIMULATION BY SELECTIVELY REDUCING THE ACTIVITY OF THE SYMPATHETIC NERVOUS SYSTEM 18039

(71) Applicant: University of Iowa Research Foundation, Iowa Cit, IA (US)

(72) Inventors: Marshall T. HOLLAND, Iowa City, IA (US); Gary PIERCE, Iowa City, IA (US); Seth W. HOLWERDA, Iowa City, IA (US); Chandan G. REDDY, Gainesville, FL (US)

(21) Appl. No.: 16/980,259

(22) PCT Filed: Mar. 14, 2019
(86) PCT No.: PCT/US2019/022314

§ 371 Date: Sep. 11, 2020

Related U.S. Application Data
(60) Provisional application No. 62/642,771, filed on Mar. 14, 2018.
(60) Provisional application No. 62/749,162, filed on Oct. 23, 2018.

(51) Int. Cl. A61N 1/36; A61N 1/05; A61N 1/20

(52) U.S. Cl. CPC A61N 1/36062; A61N 1/205; A61N 1/0551

(57) ABSTRACT

The disclosed apparatus, systems and methods relate to the use of spinal cord stimulation (SCS) in the treatment of conditions correlated to the sympathetic nervous system. Stimulating the spinal cord via SCS to decrease muscle sympathetic nerve activity (MSNA). The methods include providing acute spinal cord stimulation and selectively increasing blood flow to tissues that are physically associated with the stimulated nerves, which is used to normalize blood pressure in hypertensive or hypotensive patients. The methods establish that acute SCS rapidly increases femoral vascular conductance and reduces peroneal MSNA.

### CROSS-REFERENCE TO RELATED APPLICATION(S)

This application is a U.S. national stage application under 35 U.S.C. 371 and claims priority to International PCT Application No. PCT/US19/22314 filed Mar. 14, 2019, which claims priority to U.S. Provisional Application No. 62/642,771 filed Mar. 14, 2018 and entitled “Treatment Using Spinal Cord Stimulation,” and U.S. Provisional Application No. 62/749,162 filed Oct. 23, 2018 and entitled “Systems, Devices And Methods For Treatment Using Spinal Cord Stimulation By Selectively Reducing The Activity Of The Sympathetic Nervous System,” both of which are hereby incorporated by reference in their entireties under 35 U.S.C. § 119(e).

### TECHNICAL FIELD

The disclosed technology relates generally to spinal cord stimulation and increasing blood flow to tissues.

### BACKGROUND

The autonomic nervous system is partially comprised of sympathetic nerves that are present in the spinal cord and exit the spinal cord through various ganglions. The sympathetic nervous system is continually active at a basic level to maintain homeostasis of blood pressure, blood flow to the limbs and organs, and body temperature. The sympathetic nervous system is also known for its control of the flight or fight response in which the body is quickly prepared for responses to perceived dangers and threat.

A particular function of the sympathetic nervous system is control of blood flow to the limbs. Increased blood flow by reductions in sympathetic nerve activity to the limbs is important in cases of wound healing and ischemic pain. Complications arise when wounds do not heal and this increases the risk of additional medical problems. Patients that are in otherwise compromised states of physical health, for example, due to diabetes, cancer therapeutic treatments, or traumatic injury, often do not have a normal wound healing response.

Sympathetic nerve activity is also important in blood pressure regulation. An increase in blood flow in the limbs indicates reduced peripheral resistance of the vasculature which promotes a reduction in blood pressure. Therefore, lower sympathetic nerve activity causes a reduction in blood pressure, which is important for treatment of hypertension (high blood pressure). It is estimated that 103 million adults (almost half of all adults) in the U.S. have high blood pressure, and the prevalence of high blood pressure is expected to continue to rise. A growing percentage of hypertensive patients do not respond to current pharmacological treatments. Studies indicate that 1 in 50 patients beginning antihypertensive treatment would need 4 or more medications within 1.5 years of follow-up. Discovering therapeutic modalities that target the sympathetic nervous system to promote reduced blood pressure is paramount to health care.

Accordingly, there is a clear need for an improved method for increasing blood flow to the limbs for accelerating and fostering healing of a subject's wounds and/or reducing ischemic pain, particularly those wounds that prove to be resistant to other more conventional treatments. There is also a clear need for more efficacious methods of reducing blood pressure beyond the pharmacological and few interventional treatments that are currently available.

### BRIEF SUMMARY

Discussed herein are various devices, systems and methods relating to the use of spinal cord stimulation for the decrease of sympathetic nervous system in patients in need of treatment. In certain implementations these devices, systems and methods acutely increase blood flow to peripheral tissues.

The devices, systems and methods include stimulating the spinal cord via spinal cord stimulation (SCS) and decreasing muscle sympathetic nerve activity (MSNA). The methods include stimulation of the spinal cord such that it causes a decrease in muscle sympathetic nerve activity. In some embodiments the methods include providing acute spinal cord stimulation and selectively increasing blood flow to tissues that are physically associated with the stimulated nerves. Selectively increasing blood flow to tissues refers to, for example, stimulating the spinal cord in the lower back or lower limbs and observing an increase in femoral artery blood flow, but yet not observing a similar increase in the brachial artery blood flow. Thus, allowing for selective increases in blood flow such that wounded tissue requiring increased blood flow can be targeted. Additional methods described herein relate to using chronic SCS to treat resistant autonomic overactivity and/or resistant central neurogenic hypertension. Current therapies for these diseases include medications (typically addressing end-organs), renal denervation, and carotid body stimulation.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One Example is directed to a method for selectively reducing the activity of the sympathetic nervous system in a patient in need thereof, including: implanting a pulse generator in the patient, the pulse generator having one or more leads; and stimulating one or more nerves associated with the spinal cord of the patient.

Implementations of this Example may include one or more of the following features. The Example method further including selectively stimulating the nerve at an intensity for a duration such that blood flow is increased to tissue innervated by the nerve. The Example method where the tissue is an artery. The Example method where the one or more nerves are physically associated with the tissue. The Example method further including selectively stimulating the nerve at an intensity for a duration such that blood pressure is normalized. The Example method where the one or more nerves are sympathetic nerves. The method where the patient is diabetic. The Example method where the patient is hypertensive. The Example method where the patient is undergoing an anti-hypertensive medication regimen. The Example method where the patient has resistant autonomic overactive hypertension or resistant central neurogenic hypertension. The Example method where stimulation is applied at voltages between about 1.0v and about 10.0v. The Example method where the stimulation is applied at pulse widths ranging from about 40 μs to about 1000 μs. The Example method where the stimulation is applied at frequencies from about 20 hz to about 400 hz. Implementations of the described techniques may include hardware, a method or process, or computer software on a computer-accessible medium.

Another Example relates to a method for selectively reducing the activity of the sympathetic nervous system in a hypertensive patient, including: implanting a pulse generator in the patient, the pulse generator having one or more leads; and stimulating one or more nerves associated with the spinal cord of the patient, where the nerve stimulation reduces patient blood pressure.

Implementations according to these Examples may include one or more of the following features. The Example method where the patient is undergoing an anti-hypertensive medication regimen. The Example method where the patient has resistant autonomic overactive hypertension or resistant central neurogenic hypertension.

Another Example relates to a method of selectively reducing muscle sympathetic nerve activity in a patient, including: implanting a pulse generator in the patient, the pulse generator having one or more leads; and stimulating one or more nerves associated with the spinal cord of the patient, where the nerve stimulation increases femoral vascular conductance.

Implementations of this Example may include one or more of the following features. The Example method where the patient has neuropathic pain. The Example method where the patient is diabetic. The Example method where the femoral artery conductance is elevated over brachial artery conductance.

Implementations of the described techniques may include hardware, a method or process, or computer software on a computer-accessible medium. While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a schematic diagram of an epidural spinal cord stimulation system, according to one implementation.

FIG. 2 depicts an x-ray representation of an implanted spinal cord stimulation system within a subject, according to one implementation.

FIG. 3 shows the percent change in femoral and brachial artery conductance (FIG. 3A) and individual values for absolute femoral artery blood flow (FIG. 3B) during spinal cord stimulation (SCS, n=12) and 15-min post SCS (n=9) for Example 1, as well as the percent change in muscle sympathetic nerve activity (MSNA) burst incidence (FIG. 3C) and individual values for MSNA burst incidence (FIG. 3D) during SCS (n=5) and 15-min post SCS (n=3) in Example #2. * Significantly different from baseline, # significantly different from brachial artery, t significantly different from 15 min.

FIG. 4 depicts individual muscle sympathetic nerve activity (MSNA) recordings in 3 spinal cord stimulation (SCS) patients at baseline, during SCS, and 15 min post SCS. MSNA recording was not able to be collected 15 min post SCS in patient 4 to observe recovery of MSNA burst incidence and amplitude; however, peak MSNA burst amplitude in response to end-expiratory breath hold was recorded to display that quality of the MSNA signal during SCS was maintained (FIG. 4H).

FIG. 5 depicts the systolic blood pressure (FIG. 5A), diastolic blood pressure (FIG. 5B), and mean blood pressure (FIG. 5C) in stage 1 hypertensive (HTN) and stage 2 HTN patients before (Pre-SCS) and during intermittent epidural spinal cord stimulation (SCS). # P<0.05 vs. Pre-SCS; * P<0.05 vs. stage 1 HTN.

FIG. 6 depicts the systolic blood pressure (FIG. 6A), diastolic blood pressure (FIG. 6B), and mean blood pressure (FIG. 6C) in hypertensive (HTN) patients that were or were not taking anti-HTN medications when receiving intermittent epidural spinal cord stimulation (SCS). # P<0.05 vs. Pre-SCS; * P<0.05 vs. no HTN meds.

FIG. 7 depicts a histogram showing the neuropathic pain in stage 1 hypertensive (HTN) and stage 2 HTN patients before (Pre-SCS) and during intermittent epidural spinal cord stimulation (SCS) (FIG. 7A), in in HTN patients that were or were not taking anti-HTN medications when receiving SCS implant (FIG. 7B). # P<0.05 vs. Pre-SCS.

FIG. 8 depicts a partial correlation between mean arterial pressure (MAP) before epidural spinal cord stimulation (Pre-SCS) and change in MAP following SCS implant in hypertensive (HTN) patients controlling for sex, age, number of anti-HTN medications, and change in neuropathic pain score with SCS. Open circles: no anti-HTN medications; Closed circles: receiving ≥1 anti-HTN medication(s) at time of SCS implant. Data values are weighted for total count of blood pressure measurements. R=−0.35, P<0.0001.

FIG. 9 shows three histograms demonstrating a greater reduction in arterial blood pressure in hypertensive patients (HTN) with chronic pain compared with non-hypertensive patients (nonHTN) with chronic pain following implant of epidural spinal cord stimulation. Reduction in systolic blood pressure (FIG. 9A)), diastolic blood pressure (FIG. 9B), and mean arterial blood pressure (FIG. 9C) in non-hypertensive (nonHTN) and medicated hypertensive (HTN) patients with chronic pain following implant of epidural spinal cord stimulation.

FIG. 10 shows histograms demonstrating neuropathic pain scores in patients with chronic pain and stage 1 or stage 2 hypertension (HTN) compared with in patients with chronic pain that are non-hypertensive (nonHTN) before (Pre-SCS) and during intermittent epidural spinal cord stimulation (SCS) (FIG. 10A). FIG. 10B shows the change (delta) in neuropathic pain in these patients. # P<0.05 vs. Pre-SCS. FIG. 10C depicts the correlational analysis between change in mean arterial pressure (MAP) following implant of SCS and the change in pain score (scale 1-10) that suggests no significant relation between the change in MAP and pain in these patients.

### DETAILED DESCRIPTION

The various embodiments disclosed or contemplated herein relate to devices, systems and methods for the use of spinal cord stimulation (SCS) in subjects in need thereof.

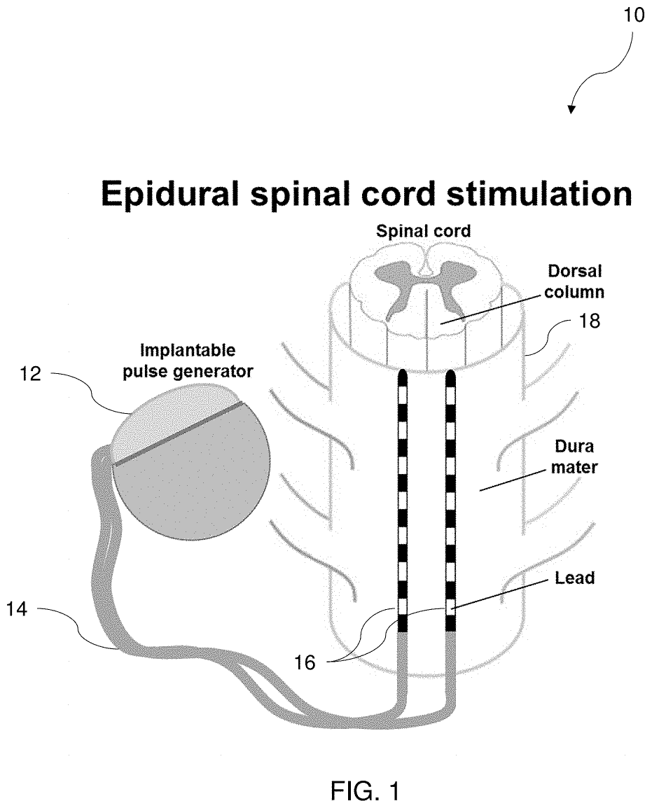

As shown in FIG. 1, in one implementation of an SCS system 10, a pulse generator 12 is implanted in the subject or patient, the pulse generator having a plurality of wired connections 14 putting it in operable and electrical communication with one or more leads 16 that are placed on the spinal column 18, such as on the dura matter surrounding the dorsal portion of the spinal cord 18. It is understood that in various implementations, the placement of the leads can be oriented more rostrally or caudally, depending on the specific application and the neural tissue or nerves of interest which may include the spinal cord itself, spinal cord tracts, spinal cord rootlets, and peripheral nerves. It is understood that references to nerves herein are meant to contemplate all such neural tissue.

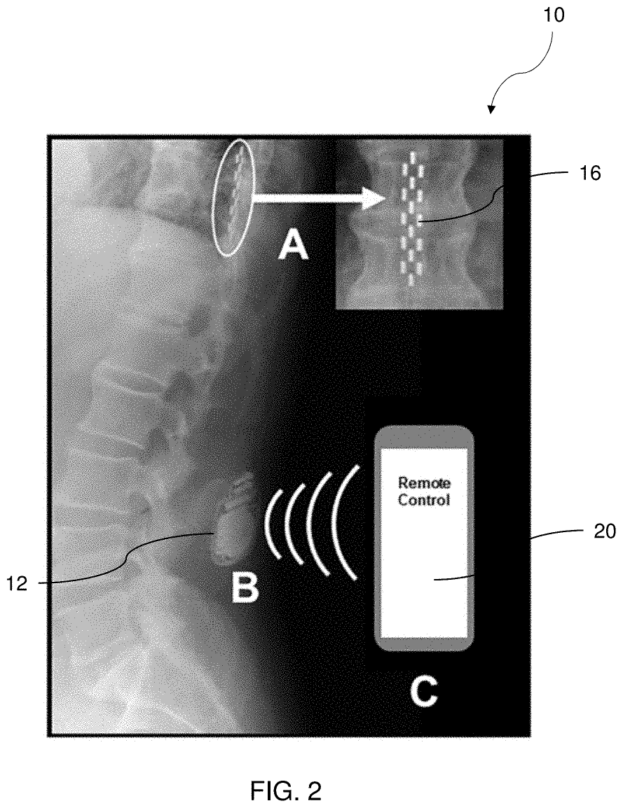

FIG. 2 depicts an implementation of the system 10 implanted in a patient. In this implementation, a remote control 20 such as a mobile device is in operational communication with the pulse generator 12 so as to control the application of electricity via the leads. Many alternate implementations are understood to be contemplated.

In various implementations, the pulse generator 12 is implanted into a patient via any known or recognized surgical technique. The leads 16 in communication with the pulse generator 12 via wired connections 14 are placed in electrical communication with various portions of the spinal column 18. The pulse generator 12 is constructed an arranged such that electrical pulses generated in the pulse generator 12 can be communicated to the spinal column 18 via wired connections 14 and the leads 16 thereby stimulating one or more neural tissues or nerves of the patient.

The system 10, in various implementations, is constructed and arranged such that the neural tissue or nerves of the patient are stimulated at an intensity for a duration such that blood flow is increased to tissue innervated by the neural tissue or nerve. In these and alternate implementation, the system 10 is constructed and arranged such that the neural tissue or nerves of the patient are stimulated at an intensity for a duration such that blood pressure is normalized. Various combinations of intensity and duration may be used.

Stimulation of the neural tissue or nerve(s) via the system 10 may reduce patient blood pressure. Additionally, stimulation of the neural tissue or nerve(s) via the system 10 may increase femoral vascular conductance. The stimulation of the neural tissue or nerve(s) via the system 10 may elevate femoral artery conductance over brachial artery conductance.

In some implementations the neural tissue or nerve(s) to be stimulated is a sympathetic nerve. In these and other implementations, the neural tissue or nerve(s) that is stimulated via the pulse generator 12 is physically associated with the tissue. In some implementations the tissue innervated by the nerve(s) or neural tissue is an artery. Use of the system 10 with other types of nerves, neural tissue and tissues is of course possible.

Various implementations address the needs of a patient. In various implementations, the system 10 can be used in connection with a patient that is diabetic and/or hypertensive. A patient may be in need of stimulation because of hypertension. A patient may be in need of SCS because of hypotension. A patient may be in need of blood pressure normalization. A patient may be in need of pain management. A patient may be in need of increased blood flow. The system 10 may also, in further implementations, be used with a patient that has resistant autonomic overactive hypertension and/or resistant central neurogenic hypertension. The system 10 may be used with a patient that has neuropathic pain. The system 10 may be used in conjunction with or instead of an anti-hypertensive medication regimen for the patient.

It is understood that in a variety of implementations, several different lead types, such as paddle, percutaneous and other leads can be utilized and placed at variety of cervical, thoracic, lumbar, sacral or other locations.

These various leads 16 can be operated at a variety of parameters such as voltages between less than about 1.0V and about 10.0V or more, with pulse widths ranging from less than about 40 μs to 1000 μs or more, and at frequencies from less than about 20 Hz to about 400 Hz or more. The treatment of a patient in need thereof may occur for about 5 minutes, about 10 minutes, about 15 minutes, about 20 minutes, about 30 minutes, about 45 minutes, about an hour, and further less acute treatment regimens of about 90 minutes or more may be used in certain implementations. In additional implementations, durations of days, weeks or months may also be employed as would be apparent to one of skill in the art from the Examples described herein. Any and all combinations of voltage, frequency, pulse widths and durations that are well-understood in the art can be employed. Those of skill in the art will appreciate that further implementations are of course possible.

The various applications of these implementations are discussed in the following experimental examples.

### EXPERIMENTAL EXAMPLES

The following examples are put forth so as to provide those of ordinary skill in the art with a complete disclosure and description of how the articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Example 1 tested the hypothesis that acute SCS (<60 min) of the thoracic spine would lead to increased common femoral vascular conductance, but not brachial vascular conductance, in 12 patients that previously underwent surgical SCS implantation (2.5±1.3 years) for management of neuropathic pain. During SCS, common femoral artery conductance was elevated and significantly greater than brachial artery conductance throughout 60 min of SCS (15 min: Δ24±10% vs. −1±5%; 30 min: 26±13% vs. −1±7%; 45 min: 43±13% vs. −1±6%; 60 min: 33±17% vs. 1±7%; 15 min post SCS: 51±21% vs. 4±10% ml/min, P=0.010).

Example 2 further examined whether acute SCS reduces peroneal MSNA in a subset of the SCS patients (n=5). MSNA burst incidence in response to acute SCS gradually declined and was significantly reduced at 45- and 60-min of SCS (15 min: Δ−1 ±6%, 30 min: Δ−14±5%, 45 min: Δ−19±7%, 60 min: Δ−24±8%, 15 min post SCS: Δ−11±3% bursts/100heartbeats, P=0.015). These data demonstrate that acute SCS rapidly increases femoral vascular conductance and reduces peroneal MSNA. The gradual reduction in peroneal MSNA observed during acute SCS suggests that neural mechanisms in addition to attenuated MSNA may be involved in the acute rise in femoral vascular conductance.

Example 3 explores the impact of SCS on hypertensive (HTN) patients to assess the hypothesis that the application of SCS will normalize blood pressure (BP) in these subjects, particularly in subjects engaging in anti-HTN medication regimes. Evidence suggests that epidural spinal cord stimulation (SCS) reduces peripheral sympathetic outflow in patients treated for peripheral artery disease and neuropathic pain. However, the extent to which SCS reduces arterial blood pressure (BP) has received little attention. This Example establishes that SCS treatment for neuropathic pain in HTN patients reduces BP independent of reductions in pain, and that the reduction in BP may be potentiated by anti-hypertensive medications.

In the present study, we examined whether acute SCS (60 min) significantly increases femoral vascular conductance in patients that previously received surgical SCS implantation for the purpose of neuropathic pain management. Because SCS implants were at the level of the thoracic spine, we hypothesized that acute SCS would increase femoral vascular conductance, but not brachial vascular conductance. In addition, to examine whether increased femoral vascular conductance following acute SCS can be associated with decreased MSNA, we performed a second Example study in a subset of the SCS patients in which MSNA was measured in response to acute SCS. Finally, we examined the effects of SCS on hypertensive patients, on or off anti-hypertensive medication regimens.

### EXPERIMENTAL METHODS AND MEASUREMENTS

Ethical approval. All procedures for Examples 1 and 2 were approved by the Institutional Review Board at the University of Iowa (Project#: 201605777). Each participant received a verbal and written explanation of the Example study's objectives, measurement techniques, and risks and benefits associated with the investigation prior to providing written informed consent.

Subjects. In Examples 1 and 2, twelve patients (54±12 years, 5 men/7 women) that previously underwent SCS implantation (2.5±0.4 years) to treat neuropathic pain originating in the lower back and/or limbs were recruited through the Department of Neurosurgery at the University of Iowa Hospitals and Clinics. Subject characteristics are presented in Table 1. With each patient's condition in mind, patients were studied and evaluated in protocol 1 (vascular conductance responses to 60 min of SCS), and based on the level of ease in which protocol 1 was performed, patients returned to participate in protocol 2 (MSNA responses to 60 min of SCS). Patients were excluded from these Examples if previously diagnosed or suffered from peripheral vascular disease, heart disease, renal failure, type 1 diabetes, cancer, autonomic dysfunction, have had history of sympathectomy or any form of heart surgery.

In Example 3, Review of medical records (n=611) from the years 2008 to 2018 from patients that underwent surgical implantation of SCS to treat neuropathic pain originating in the lower back and/or limbs at the Department of Neurosurgery at the University of Iowa Hospitals and Clinics. 504 patients had clinic BP records within two years prior and within two years after surgical implantation of SCS. Patients were categorized as stage 1 HTN (SBP 130 or DBP 80 mmHg, n=170), or stage 2 HTN (SBP 140 or DBP 90 mmHg, n=141) based on records prior to surgery. It was further hypothesized that BP reduction following SCS would be augmented in patients taking anti-HTN medications; therefore, all types of anti-HTN medications that patients were receiving at the time of surgical implant of the SCS device was documented for analysis.

Epidural dorsal column stimulation. The spinal cord stimulator (SCS) system 10, like that shown in FIG. 1 and FIG. 2, consisted of a lead wire 14 with up to sixteen electrodes implanted posterior of the dorsal column of the spinal cord, and a pulse generator 12 with a rechargeable battery to deliver electrical current. The SCS device is controlled via a hand-held wireless remote (as is shown in FIG. 2 at 20), which has the capability to turn the device on and off and adjust programs that determine the level of stimulation. Individual patient electrode implant locations and SCS programming configurations are summarized in Table 2.

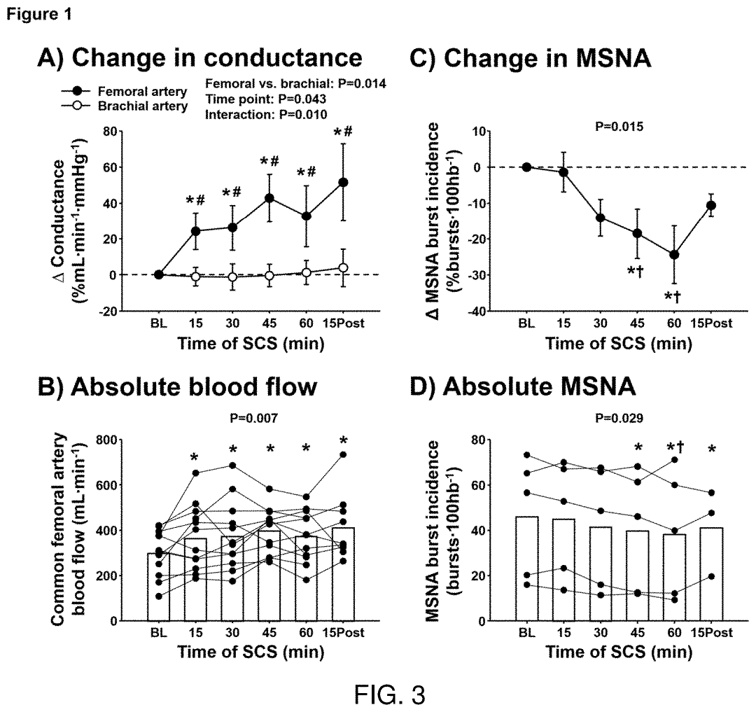

Muscle sympathetic nerve activity. Multi-unit MSNA was recorded using understood microneurographic techniques (FIG. 3C). Briefly, a tungsten microelectrode was placed into the peroneal nerve near the left fibular head. Signals were amplified, filtered (bandwidth 0.7-2.0 kHz), rectified and integrated (0.1 s time constant) to obtain mean voltage neurograms (Nerve Traffic Analyzer 662c-3; University of Iowa Bioengineering, Iowa City, Iowa). MSNA data was acquired at a frequency of 1,000 Hz using a Powerlab data acquisition system (ADlnstruments) and analyzed using LabChart version 8.1.5 (ADInstruments). MSNA was identified by the presence of spontaneous bursts with characteristic pulse synchronicity and by its responsiveness to end-expiratory breath holds, but not to arousal or skin stimulation.

Vascular conductance: Common femoral artery blood velocity and diameter were determined with a 12-MHz linear-array Doppler probe (Logiq7, GE Healthcare, Inc.) on the right common femoral artery approximately 2-3 cm proximal of the bifurcation. Common femoral and brachial artery blood velocity was measured continuously for 2 min at baseline and at each time point during SCS (15, 30, 45, and 60 min), and at 15 min post SCS, with a probe insonation angle of 60 degrees. Blood flow was calculated as the product of mean blood velocity (cm/s) and cross-sectional area (cm2) and multiplied by 60 (milliliters per minute). Brachial artery blood flow on the right arm at heart level was measured approximately 2-3 cm proximal of the antecubital fossa at the same time points as femoral artery blood flow. The order of femoral and brachial artery blood flow measures for each patient was randomized. Femoral and brachial artery blood flow was divided by mean blood pressure (automated sphygmomanometer on the left arm) to derive vascular conductance.

Data Analysis: Femoral and brachial vascular conductance and MSNA were calculated as percent change from baseline. Absolute values of femoral vascular conductance and MSNA are also presented. MSNA was quantified as burst incidence (bursts/100heartbeats), burst frequency (bursts/min), and total activity (burst frequency multiplied by mean burst amplitude, arbitrary units/min). MSNA burst amplitude was calculated by attributing the value of 100 to the maximum burst height during the baseline recording, which was determined from the average of the 3 largest bursts, and expressing all other burst amplitudes as a percentage of the maximum burst height as previously described (Holwerda et al., 2016a, Vranish et al., 2018, Fairfax et al., 2013).

Changes in blood flow, HR, BP, MSNA and neuropathic pain ratings during SCS were examined using repeated measures ANOVA. Comparison of changes in femoral and brachial artery blood flow during SCS was examined using two-way repeated measures ANOVA. Where there were missing data after 60 min of SCS (15 min post-SCS) for measures for blood flow (n=3) and MSNA (n=2), the estimate of these values was calculated as the marginal sums of squares (Type III, adjusted sums of squares) using a general linear model approach. Fisher LSD post-hoc analysis was used where significant main effects were observed. Statistical analyses were performed using SigmaPlot version 13.0 (Systat Software, Inc.). Data are presented as mean±SEM.

### EXPERIMENTAL PROTOCOLS

Example 1: Femoral and brachial vascular conductance responses to acute SCS. SCS Example patients arrived at the University of Iowa Clinical Research Unit following an overnight fast (n=12). Example patients were instructed to deactivate their SCS unit and abstain from medications for 48 hours prior to the Example visit, and to abstain from caffeine the morning of the Example. Example visits were initiated between 7:00 am and 9:00 am, and were performed in a quiet, dimly lit, temperature-controlled room (21-22° C.). After a 15-min rest period, patients were instrumented for heart rate (HR), blood pressure (BP, arm cuff), and femoral and brachial artery blood flow. Following baseline measures of femoral and brachial artery blood flow, the SCS unit was activated via remote control at the individual patient's clinically prescribed level of stimulation (see Table 1), and femoral and brachial artery blood flow was measured every 15 min during SCS (60 min) and 15-min post SCS. The SCS unit can be sensed by patients when functioning (typically described as a paresthesia or vibration sensation), therefore a sham condition in the Example protocol was precluded. Visual analogue score of neuropathic pain on a scale of 0-10 was reported by the patient at each time point.

Example 2: Influence of acute SCS on muscle sympathetic nerve activity. Within 1-3 weeks of Example 1, SCS patients returned to the University of Iowa Clinical Research Unit following an overnight fast (n=5). The remaining 7 patients from Example 1 are not included in Example 2 because their conditions precluded them from resting comfortably and still for stable 60-min peroneal MSNA recordings. Study patients for Example 2 were instructed to deactivate their SCS unit and abstain from medications for 48 hours prior to the study visit, and to abstain from caffeine the morning of the study. Patients were instrumented for HR, BP, and MSNA after a 15-min rest period. Following a 10-min baseline, the SCS unit was activated and remained active for 60 min at the individual patient's prescribed level of stimulation. MSNA, HR and BP were measured for a 5-min period every 15 min during SCS, and 15-min post SCS. Post-SCS recordings of Visual analogue score of neuropathic pain on a scale of 0-10 was reported by the patient at each time point.

Example 3: Hypertension. Example 3 tested the hypothesis that mean BP (MAP) would be reduced in HTN patients receiving surgically implanted SCS for management of neuropathic pain, and that MAP reduction would be independent of reduced neuropathic pain. It was further hypothesized that MAP reduction following SCS would be augmented in patients taking anti-HTN medications. Event-triggered averaging was used to determine 2-year BP averages prior to SCS implant (pre-SCS) and 2-year BP averages after SCS implant (SCS). Records of neuropathic pain intensity, as determined by visual analogue scale (0-10, 0=no pain, 10=worst pain possible), were also examined to determine the extent of pain reduction following surgery and the relation to change in BP.

Retrospective analysis of daytime MAP in patient medical records was performed from 2008 to 2018 and averaged within 2 years prior to SCS implant (pre-SCS) and compared with MAP averaged within 2 years after SCS implant (SCS) in stage 1 HTN (n=170) and stage 2 HTN (n=141) patients.

Example 4: Elevation. Example 4 tested the hypothesis that mean BP (MAP) would be elevated in HTN patients with low or normal BP receiving surgically implanted SCS for management of neuropathic pain, and that the elevation in MAP would be independent of reduced neuropathic pain.

The retrospective cohort study used in Examples 3-4 was approved by the Institutional Review Board at the University of Iowa (Project#: 201805969). Review of medical records was performed on 611 patients from the Department of Neurosurgery at the University of Iowa Hospitals and Clinics that underwent surgical implantation of SCS to treat neuropathic pain originating in the lower back and/or limbs from the years 2008 to 2018. Of the 611 patients reviewed, 504 patients had clinic BP records within two years prior and within two years after surgical implantation of SCS. Patients were categorized as non-hypertensive (n=193), stage 1 HTN (SBP 130 or DBP 80 mmHg, n=170), or stage 2 HTN (SBP 140 or DBP 90 mmHg, n=141) based on clinic BP records prior to surgery. Records of neuropathic pain intensity, as determined by visual analogue scale (0-10, 0 =no pain, 10=worst pain possible), were collected within two years prior and within two years after surgical implantation of SCS to determine the extent of pain reduction following surgery and the relation to change in BP.

### EXAMPLES

### Example 1: Vascular Conductance

Vascular conductance responses to acute SCS. The change in femoral vascular conductance was significantly elevated and greater than brachial vascular conductance at 15-, 30-, 45-, and 60-min of SCS, and 15-min post SCS (Artery×time point interaction: P=0.010) (FIG. 3A). Despite the absence of stimulation at 15 min post SCS, femoral vascular conductance remained elevated compared to brachial vascular conductance. Femoral artery blood flow was elevated and significantly increased at 30- and 45-min (P=0.016) (FIG. 3B). No changes in end-diastolic femoral artery diameter were detected during SCS (BL: 7.9±1.9, 15 min: 7.9±1.9, 30 min: 7.8±2, 45 min: 8±1.8, 60 min: 8.1±1.8 mm, P=0.215). Blood flow after 60 min of SCS (15 min post-SCS) was not able to be collected on patients 2, 4 and 5 because of the inability to remain rested in the supine position.

Cardiovascular responses to acute SCS. No changes in HR were observed throughout SCS (BL: 60±6, 15 min: 59±7, 30 min: 60±8, 45 min: 60±8, 60 min: 61±8, 15 min post: 60±6 bpm, P=0.778). Although there were no significant changes in systolic BP (P=0.086) or diastolic BP (P=0.133) during SCS, there was a small but significant increase in mean BP (BL: 87±10, 15 min: 90±11, 30 min: 92±11, 45 min: 90±12, 60 min: 91±12, 15 min post: 87±13 mmHg, P=0.012). The increase in mean BP was consistent among patients. As expected, self-reported neuropathic pain was reduced following SCS and remained reduced throughout SCS (scale 0-10, BL: 3.5 ±3.3, 15 min: 1.7±2.2, 30 min: 1.6±1.9, 45 min: 1.6±2.1, 60 min: 1.6±2.1, 15 min post: 2.2±3.0, P=0.003).

### Example 2: Muscle Sympathetic Nerve Activity (MSNA)

Influence of acute SCS on muscle sympathetic nerve activity: MSNA during SCS gradually declined and became significantly reduced at 45- and 60-min of SCS compared with baseline and 15 min (P=0.015) (FIG. 3C). Similar results were observed for the change in MSNA burst frequency (15 min: Δ−1±6%, 30 min: Δ−13±4%, 45 min: Δ−17±6%, 60 min: Δ−21±7%, 15 min post: Δ−4±5% bursts/min, P=0.027). Absolute values of MSNA burst incidence were significantly reduced compared to baseline at 45- and 60-min of SCS, and remained attenuated 15 min post-SCS (FIG. 3D).

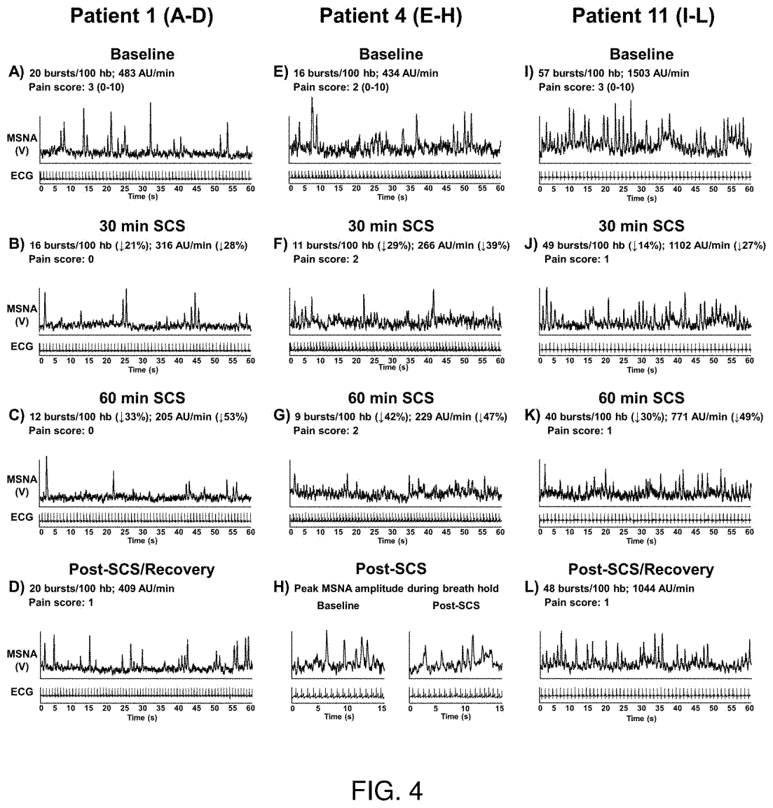

Individual MSNA recordings showing burst incidence and total activity during SCS from 3 patients are presented in FIG. 4. MSNA recording did not continue after 60 min of SCS (15 min post-SCS) in patients 4 because of the inability to remain in the fixed position required to maintain the MSNA recording. However, an end-expiratory breath hold was performed by patient 4 immediately following the cessation of SCS, and the peak MSNA amplitude response was comparable to the response at baseline (FIG. 4H), confirming that the reduction in MSNA during SCS was not a result of deterioration in the MSNA signal. Recording of MSNA after 60 min of SCS (15 min post-SCS) was also not able to be collected in patient 2. When comparing changes in MSNA during SCS in Example 2 to changes in the same patients' femoral artery conductance during Example 1, there was no apparent relation because MSNA was gradually reduced while femoral artery conductance rapidly increased.

Cardiovascular responses to acute SCS: Similar to Example 1, no change in HR was observed during SCS (BL: 63±4, 15 min: 64±4, 30 min: 64±4, 45 min: 64 ±4, 60 min: 65±4, 15 min post: 67±6 bpm, P=0.197). Also, no significant changes in systolic BP (P=0.773) and diastolic BP (P=0.598) were observed. Mean BP during SCS was not significantly different compared to baseline (BL: 90±7, 15 min: 91±11, 30 min: 92±10, 45 min: 91±11, 60 min: 93±11, 15 min post: 86±14 mmHg, P=0.197), although the pattern tended to be similar to Example 1. Neuropathic pain appeared reduced within the first 15 min and remain unchanged thereafter, although not reaching statistical significance (scale 0-10, BL: 2.2±1.3, 15 min: 0.8±0.8, 30 min: 0.6±0.9, 45 min: 0.8±1.1, 60 min: 0.6±0.9, 15 min post: 1.8±1.9, P=0.098).

### Examples 3 & 4: Blood Pressure Normalization

Evidence suggests that epidural spinal cord stimulation (SCS) reduces peripheral sympathetic outflow in patients treated for peripheral artery disease and neuropathic pain. However, the extent to which SCS reduces arterial blood pressure (BP) has received little attention. In this Example, the effects of SCS on hypertensive (HTN) patients is assessed.

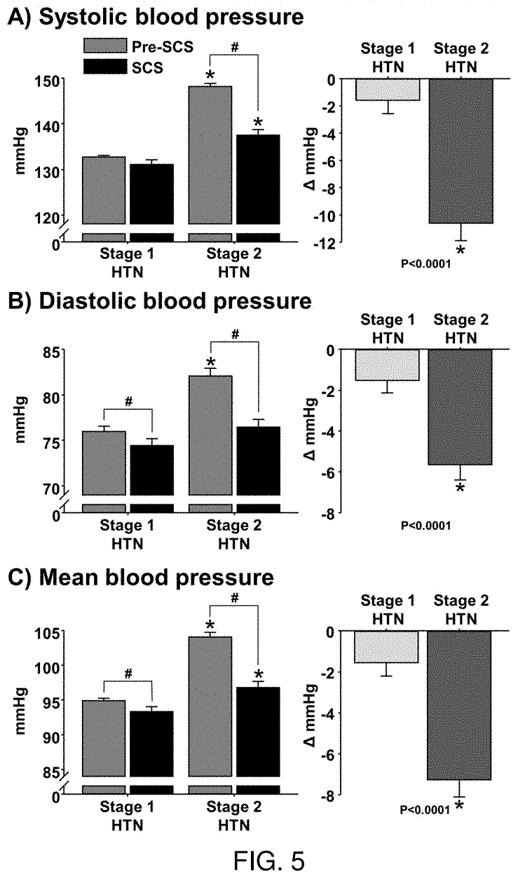

FIGS. 5A-5B depict the systolic blood pressure (FIG. 5A), diastolic blood pressure (FIG. 5B), and mean blood pressure (FIG. 5C) in stage 1 hypertensive (HTN) and stage 2 HTN patients before (Pre-SCS) and during intermittent epidural spinal cord stimulation (SCS). # P<0.05 vs. Pre-SCS; * P<0.05 vs. stage 1 HTN.

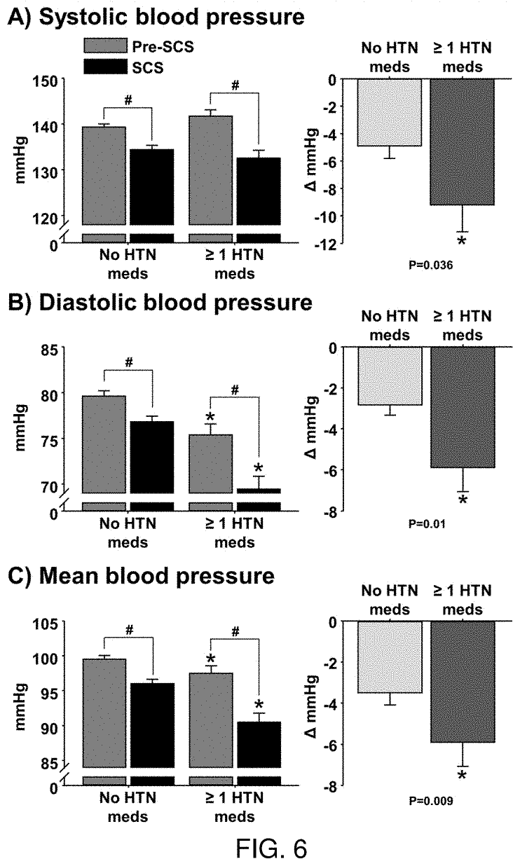

FIG. 6A-6C depict the systolic blood pressure (FIG. 6A), diastolic blood pressure (FIG. 6B), and mean blood pressure (FIG. 6C) in hypertensive (HTN) patients that were or were not taking anti-HTN medications when receiving intermittent epidural spinal cord stimulation (SCS). # P<0.05 vs. Pre-SCS; * P<0.05 vs. no HTN meds.

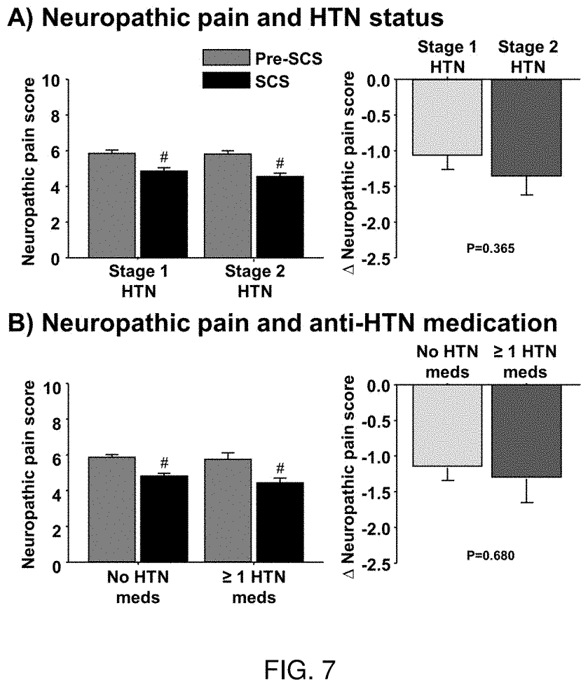

FIGS. 7A-7B are histograms showing the neuropathic pain in stage 1 hypertensive (HTN) and stage 2 HTN patients before (Pre-SCS) and during intermittent epidural spinal cord stimulation (SCS) (FIG. 7A), in in HTN patients that were or were not taking anti-HTN medications when receiving SCS implant (FIG. 7B). # P<0.05 vs. Pre-SCS.

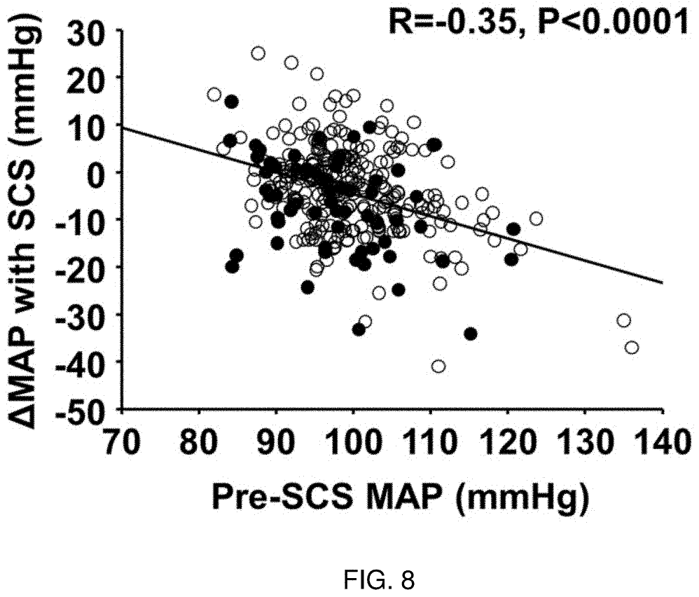

FIG. 8 depicts a partial correlation between mean arterial pressure (MAP) before epidural spinal cord stimulation (Pre-SCS) and change in MAP following SCS implant in hypertensive (HTN) patients controlling for sex, age, number of anti-HTN medications, and change in neuropathic pain score with SCS. Open circles: no anti-HTN medications; Closed circles: receiving 1 anti-HTN medication(s) at time of SCS implant. Data values are weighted for total count of blood pressure measurements. R=−0.35, P<0.0001.

Hypertension. SCS MAP was significantly lower compared with pre-SCS MAP (Pre-SCS: 99 vs. SCS: 95 mmHg, P<0.001), and the reduction was greater in stage 2 HTN compared with stage 1 HTN (Stage 2: −7±1 vs. Stage 1: −2±1 mmHg, P<0.001), while reduction in neuropathic pain (scale 1-10) was not significantly different between groups (Stage 2: −1.4±0.3 vs. Stage 1: −1.1±0.2, P=0.37). Reduction in MAP following SCS was significantly greater in HTN patients on anti-HTN medications compared with no anti-HTN medications (Meds: −7±1 vs. No meds: −4±1 mmHg, P=0.009). Partial correlation revealed that greater reduction in MAP following SCS was significantly correlated with higher Pre-SCS MAP after controlling for sex, age, number of anti-HTN medications, and change in neuropathic pain score with SCS (R=−0.35, P<0.0001).

These findings suggest that SCS treatment for neuropathic pain in HTN patients reduces BP independent of reductions in pain, and that the reduction in BP may be potentiated by anti-hypertensive medications.

Blood Pressure Elevation. Example 4 tested the hypothesis that mean BP (MAP) would be elevated in HTN patients with low or normal BP receiving surgically implanted SCS for management of neuropathic pain, and that the elevation in MAP would be independent of reduced neuropathic pain. This analysis was performed on the same cohort as Example 3.

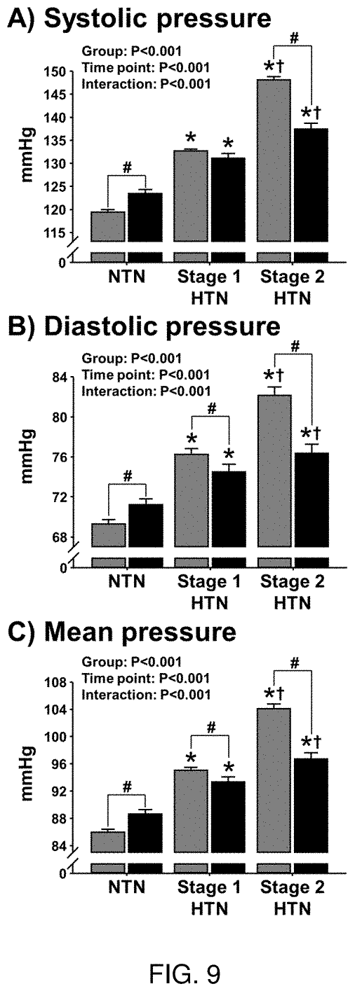

In Example 4, a greater reduction in arterial blood pressure in hypertensive patients (HTN) with chronic pain compared with non-hypertensive patients (nonHTN) with chronic pain following implant of epidural spinal cord stimulation. FIG. 9 depicts the reduction in systolic blood pressure (FIG. 9A), diastolic blood pressure (FIG. 9B), and mean arterial blood pressure (FIG. 9C) in non-hypertensive (nonHTN) and medicated hypertensive (HTN) patients with chronic pain following implant of epidural spinal cord stimulation.

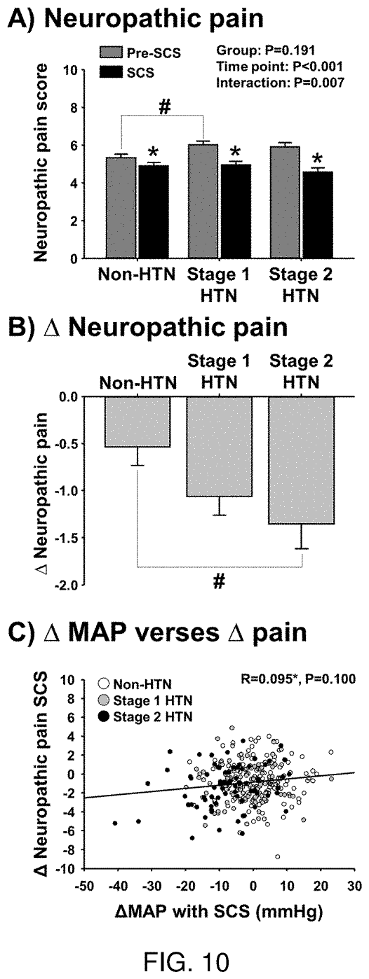

FIG. 10: Neuropathic pain scores in patients with chronic pain and stage 1 or stage 2 hypertension (HTN) compared with in patients with chronic pain that are non-hypertensive (nonHTN) before (Pre-SCS) and during intermittent epidural spinal cord stimulation (SCS) (FIG. 10A). FIG. 10B shows the change (delta) in neuropathic pain in these patients. # P<0.05 vs. Pre-SCS. FIG. 10C shows the correlational analysis between change in mean arterial pressure (MAP) following implant of SCS and the change in pain score (scale 1-10) that suggests no significant relation between the change in MAP and pain in these patients.

### DISCUSSION

The major novel findings of the present Examples are discussed herein.

First, as shown in Example 1, acute SCS significantly increases femoral vascular conductance in patients treated for neuropathic pain, while no change in brachial vascular conductance was observed. In fact, femoral vascular conductance was significantly elevated within 15 minutes of SCS, and the increase was sustained throughout SCS for 60 minutes.

Second, as observed in a subset of the SCS patients and explored in Example 2, acute SCS significantly reduced MSNA, but the reduction in MSNA was gradual and reached significance at 45 minutes of SCS. These findings demonstrate that acute SCS rapidly increases femoral vascular conductance and reduces peroneal MSNA.

Third, SCS treatment for neuropathic pain in HTN patients reduces BP independent of reductions in pain, and that the reduction in BP may be potentiated by anti-hypertensive medications, as shown in Example 3.

The influence of SCS on peripheral blood flow has been documented in several clinical studies spanning months or years of SCS treatment in patients with peripheral vascular disease. However, no studies have examined the effects of acute SCS treatment on vascular conductance in patients without history of sympathectomy and/or vascular surgery. We rationalized that acute SCS in the thoracic region of the spinal cord would lead to increased femoral vascular conductance, but not brachial vascular conductance. As hypothesized, significant increases in vascular conductance were observed in the femoral artery following SCS, whereas no change in vascular conductance was observed in the brachial artery.

Interestingly, the typical vertebral level of SCS for the purpose of increasing limb blood flow in cases of peripheral arterial disease is slightly lower (˜T9-L1) compared with the range of stimulation in present Example (˜T6-T10), although there is some overlap. Since neuropathic pain symptoms were also reduced following acute SCS, as expected, the findings of the present Example raise speculation as to whether SCS may serve multiple purposes including both neuropathic pain relief and increased lower limb blood flow. Indeed, a recent case report demonstrated simultaneous relief of neuropathic pain and Willis-Eckbom disease (restless legs syndrome), which may be associated with reduced lower limb blood flow, with SCS leads spanning the T10-T11 vertebral bodies.

Experimental animal studies have provided mechanistic evidence that SCS leads to vasodilation via reduction in sympathetic activity. It is proposed that SCS activation of afferent sensory fibers in the dorsal columns stimulate GABAergic interneurons which reduces activity of sympathetic preganglionic neurons and peripheral release of catecholamines. The present Example is the first to directly examine efferent sympathetic activity to the muscle of the lower limbs in patients with SCS. The results support and extend findings from experimental animal studies that demonstrate increased peripheral blood flow via SCS modulation of sympathetic activity.

A second theory is that SCS in the dorsal columns causes antidromic activation of small myelinated sensory fibers, leading to peripheral release of cutaneous vasodilators such as calcitonin gene-related peptide (CGRP); however, studies in animals suggest stimulation frequency higher than typically used in the clinic may be required to activate TRPV1-containing fibers and cause release of CGRP. Activation of sensory fibers by SCS and subsequent increases in cutaneous blood flow cannot be ruled out in the present Example.

Indeed, femoral vascular conductance was significantly elevated within 15 min of SCS while MSNA gradually decreased, suggesting that other neural mechanisms in addition to reduced MSNA may be contributing to elevated vascular conductance. Previous studies reported that increased cutaneous blood flow following acute SCS in patients with neuropathic pain was not a result of activation of sensory afferents, but rather an inhibitory effect on sympathetic vasoconstriction; however, studies were performed in patients with history of sympathectomy, which makes interpretation difficult. Additional studies in humans examining cutaneous blood flow during acute SCS in humans without history of sympathetctomy are needed to determine the contribution to leg vascular conductance.

Since patients studied herein were receiving SCS for neuropathic pain management, whether reduced MSNA and increased femoral vascular conductance during SCS can be explained by reduction in neuropathic pain warrants discussion. The reduction in self-reported neuropathic pain immediately followed SCS (15 min), while MSNA followed a pattern of a progressive decline, suggesting that neuropathic pain and changes in MSNA were dissociated. When considering individual responses, patient 12, for example, demonstrated 72% increase in femoral vascular conductance at 15 min while self-reported neuropathic pain at baseline was “0.” Also, self-reported neuropathic pain for patient 2 was “0” at baseline while an increase in femoral vascular conductance of 35% and a reduction in MSNA of −16% at 45 min of SCS was observed. Overall, patients did not report high levels of pain at baseline. In fact, most patients reported that normal use of SCS for neuropathic pain is in the standing position and not while lying supine. Thus, changes in neuropathic pain during SCS did not appear to explain changes in MSNA or femoral vascular conductance.

Numerous aspects of SCS influence the individualized effectiveness, including the vertebral level of the stimulation, the utilized active contacts of the electrode array, and the polarity of the active contacts which shapes the delivered current. Initial optimization of SCS settings involves interaction between the trained SCS programmer and the patient reporting areas of pain relief. As seen in Table 2, SCS parameters are highly variable between individuals. The variable SCS configurations in the present Example likely play a role in the level of MSNA inhibition and changes in vascular conductance. For example, patients 1 and 4 had percutaneous leads placed T6-T8 with different programming configurations. Patient 1 demonstrated a peak increase in femoral vascular conductance of 71% during SCS, while patient 4 demonstrated minimal changes in femoral vascular conductance. However, in Example 2, both patients 1 and 4 demonstrated a reduction in MSNA (FIG. 4).

Patient 11 had C2-C5 lead placement (the only patient with cervical lead placement) and demonstrated a minimal change in femoral vascular conductance during SCS in Example 1, but demonstrated a decrease in MSNA during SCS in Example 2. Patients 2 and 10 with percutaneous leads spanning T7-T10 had moderate increases in femoral vascular conductance during SCS in Example 1 and moderate reductions in MSNA during SCS in Example 2. These findings highlight the complexity in which SCS modulates neural activity. Importantly, in this patient population, SCS lead placement and configurations were for optimal paresthesia and pain relief, and may not have been ideal for increased leg blood flow or MSNA inhibition. In some studies of SCS for pain relief, lead type and position, and specific patient factors such as sex and age, did not show a significant effect on optimal parameter settings. The impact of such factors on MSNA inhibition and vascular conductance are less clear, particularly when considering variables such as dorsal cerebrospinal fluid layer thickness, lead migration over time, and variation in position such as supine vs. standing.

In summary, Examples 1 & 2 demonstrate for the first time in humans that acute SCS significantly increases femoral vascular conductance. In addition, peroneal MSNA was significantly attenuated in response to acute SCS. However, in contrast to the rapid rise in femoral vascular conductance, peroneal MSNA in response to acute SCS demonstrated a progressive reduction and did not appear to be directly associated with the change in femoral vascular conductance. Neural mechanisms in addition to attenuated MSNA may be involved in the initial SCS-mediated increases in femoral vascular conductance.

The results of Examples 3 & 4 suggest that SCS treatment, including when paired with an anti-HTN medication regimen, can lead to normalization of blood pressure levels in these patients. Sympathetic overactivity is characteristic of hypertension and is regarded as an underlying factor contributing to cardiovascular morbidity. Reduction in muscle sympathetic nerve activity (MSNA) is a specific target of renal denervation to produce reduction in BP in patients with resistant hypertension. Studies have reported a reduction of 19-24 mmHg in systolic BP up to one year following renal denervation. Although early clinical trials of renal denervation provided encouraging results, findings from more recent studies have been mixed and have dampened the enthusiasm for renal denervation as a modality to reduce sympathetic nerve activity and blood pressure. Results from the HOPE-3 study demonstrated that a reduction in BP of −5.8 mmHg systolic and −3.0 mmHg diastolic in persons at intermediate risk using pharmacological therapy was associated with a 28% lower incidence of major cardiovascular events compared to placebo. These findings indicate that the reduction in blood pressure observed we observe in stage 2 HTN patients with SCS treatment (−11 mmHg systolic and −6 mmHg diastolic) is clinically relevant and important. The reduction in BP with SCS treatment that we observe is primarily driven by HTN patients that are also taking anti-HTN medications, indicating a synergistic effect of SCS and anti-HTN treatment that is of interest the medical community.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed systems and methods, the subject has been diagnosed with a need for treatment prior to the treatment step.

Although the disclosure has been described with reference to preferred embodiments, persons skilled in the art will recognize that changes may be made in form and detail without departing from the spirit and scope of the disclosed apparatus, systems and methods.

TABLE 1
Subject characteristics

| | Age | Sex | SBP/DBP | |
|---|---|---|---|---|
| Patient | (years) | (M/W) | (mmHg) | Medications |
| Patient 1 | 42 | W | 109/62 | Cyclobenzaprine, Tramadol |
| Patient 2 | 75 | M | 133/68 | Lyrica, Atenolol, Tamsulosin |
| Patient 3 | 43 | W | 125/83 | HCTZ, Cyclobenzaprine, |
| | | | | Vicodin |
| Patient 4 | 36 | W | 129/75 | — |
| Patient 5 | 64 | W | 163/71 | Enalapril, Gabapentin, |
| | | | | Keppra, Oxycodone, |
| | | | | Tizanidine |
| Patient 6 | 49 | W | 113/63 | Toprol XL, Simvastatin, |
| | | | | Gabapentin, Topiramate |
| Patient 7 | 55 | W | 107/69 | Methadone |
| Patient 8 | 61 | M | 148/81 | Carvedilol, Losartan, |
| | | | | Gabapentin, Hydromorphone |
| Patient 9 | 52 | M | 124/72 | Lisinopril, Gabapentin, |
| | | | | Glipizide, Pioglitazone, |
| | | | | Atorvastatin, Tramadol |
| Patient 10 | 44 | M | 106/65 | Oxycodone, Prednisone |
| Patient 11 | 66 | M | 110/78 | Lyrica, Pramipexole, |
| | | | | Baclofen, Triamterene, |
| | | | | Simvastatin, Tramadol |
| Patient 12 | 57 | W | 96/59 | Tizanidine, Atorvastatin, |
| | | | | Estradiol |

SBP, systolic blood pressure; DBP, diastolic blood pressure.
(—) no daily medications.

What is claimed is:

1. A method for selectively reducing the activity of the sympathetic nervous system of a patient in need thereof, comprising:
a. implanting a pulse generator in the patient, the pulse generator having one or more leads; and
b. stimulating one or more nerves associated with the spinal cord of the patient.

2. The method of claim 1, further comprising selectively stimulating the nerve at an intensity for a duration such that blood flow is increased to a tissue innervated by the nerve.

3. The method of claim 2, wherein the tissue is an artery.

4. The method of claim 2, wherein the one or more nerves are physically associated with the tissue.

5. The method of claim 1, further comprising selectively stimulating the nerve at an intensity for a duration such that blood pressure is normalized.

6. The method of claim 1, wherein the one or more nerves are sympathetic nerves.

7. The method of claim 1, wherein the patient is diabetic.

8. The method of claim 1, wherein the patient is hypertensive.

9. The method of claim 8, wherein the patient is undergoing an anti-hypertensive medication regimen.

10. The method of claim 8, wherein the patient has resistant autonomic overactive hypertension or resistant central neurogenic hypertension.

11. The method of claim 1, wherein stimulation is applied at voltages between about 1.0V and about 10.0V.

12. The method of claim 1, wherein the stimulation is applied at pulse widths ranging from about 40 μs to about 1000 μs.

13. The method of claim 1, wherein the stimulation is applied at frequencies from about 20 Hz to about 400 Hz.

14. A method for selectively reducing the activity of the sympathetic nervous system in a hypertensive patient, comprising:
a. implanting a pulse generator in the patient, the pulse generator having one or more leads; and
b. stimulating one or more neural tissues associated with the spinal cord of the patient,
wherein the neural tissue stimulation reduces patient blood pressure.

15. The method of claim 14, wherein the patient is undergoing an anti-hypertensive medication regimen.

16. The method of claim 14, wherein the patient has resistant autonomic overactive hypertension or resistant central neurogenic hypertension.

17. A method of selectively reducing muscle sympathetic nerve activity in a patient, comprising:
a. implanting a pulse generator in the patient, the pulse generator having one or more leads; and
b. stimulating one or more nerves associated with the spinal cord of the patient,
wherein the nerve stimulation increases femoral vascular conductance.

18. The method of claim 17, wherein the patient has neuropathic pain or is diabetic.

19. The method of claim 17, wherein the stimulation is applied for between about 5 and about 60 minutes.

20. The method of claim 17, wherein femoral artery conductance is elevated over brachial artery conductance.
